(19) United States
(12) Patent Application Publication
(10) Pub. No.: US 2002/0018792 A1
(43) Pub. Date: Feb. 14, 2002

(54) Skin care anti-sebum compositions containing 3,4,4-trichlorocarbanilide

(75) Inventors: Bijan Steven Harichian, Warren, NJ (US); John Steven Bajor, Ramsey, NJ (US); Kathy Anne Lanza, Towaco, NJ (US); Surajit Mukherjee, Ridgewood, NJ (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 09/873,002

(22) Filed: Jun. 1, 2001

Related U.S. Application Data
(60) Provisional application No. 60/215,518, filed on Jun. 30, 2000.

(51) Int. Cl. A61K 7/00; A61K 31/165; A61K 31/07

(52) U.S. Cl. 424/401; 514/625; 514/725

(57) ABSTRACT

Anti-sebum skin care methods and compositions containing TCC. The compositions are non-greasy and aesthetically pleasing, and provide control of sebum secretion from sebocytes, improved oil control and improved skin feel, and prevent shine and stickiness.

### FIELD OF THE INVENTION

Cosmetic compositions and methods for controlling oily skin.

### BACKGROUND OF THE INVENTION

A frequent, undesirable skin condition is “oily skin,” the condition which results from the excessive amount of sebum on the skin. Sebum is skin oil which is produced by sebocytes (cells of the sebaceous glands in the skin) and is then secreted to the skin surface. Oily skin is associated with a shiny, undesirable appearance and a disagreeable tactile sensation. Oily skin affects various age groups. Cosmetic products which provide sebum control are highly desirable.

3,4,4-trichlorocarbanilide (hereinafter “TCC”) is a known anti-microbial agent and has been included as such in personal wash compositions (e.g., soap bars) and antiperspirant compositions.

### SUMMARY OF THE INVENTION

The present invention includes a leave-on skin care cosmetic composition for delivery into sebaceous glands of the skin comprising:

(i) from about 0.001% to about 10% of 3,4,4-trichlocarbanilide;

(ii) not more than 20% of an alcohol;

(iii) not more than 5% of a surfactant; and

(iv) a cosmetically acceptable vehicle,

wherein the composition has a Mean Greasy Feel Value of less than about 3.5, one minute after application to the skin and, preferably, also has a Mean Greasy Feel Value of less than about 2.0, ten minutes after application to the skin.

The present invention also includes a method of controlling or preventing an oily skin condition, especially in the facial area, by applying to the skin the inventive composition.

The invention also includes a cosmetic method of reducing, preventing or controlling sebum secretion from sebocytes by applying to the skin the inventive composition.

### DETAILED DESCRIPTION OF THE INVENTION

Except in the operating and comparative examples, or where otherwise explicitly indicated, all numbers in this description indicating amounts of material or conditions of reaction, physical properties of materials and/or use are to be understood as modified by the word “about.” All amounts are by weight of the oil-in-water emulsion, unless otherwise specified.

The term “skin” as used herein includes the skin on the face, neck, chest, back, arms, hands, legs and scalp.

The inventive methods and compositions include TCC in an amount of from 0.001% to 10%, preferably from 0.001% to 5%, most preferably from 0.001% to 2%. Preferably, the amount of TCC in the inventive compositions does not exceed 5%—using an excessive amount of TCC may be damaging to the microflora of the skin.

The inventive compositions are intended as “leave-on” compositions, i.e. they are cosmetic composition that are applied to the skin and left on it, rather than washed off.

The inventive compositions are intended for oil control and thus must themselves be non-greasy and be perceived as “non-greasy.” The non-greasiness of the composition is expressed, according to the present invention as a “Mean Greasy Feel Value” and is measured as described in Example 2 herein. The Mean Greasy Feel Value of the inventive compositions is less than 3.5 (preferably less than 3.0), when measured at one minute after application. The preferred compositions remain non-greasy, or are perceived as even less greasy after a few minutes on the skin, i.e. have a Mean Greasy Feel Value of less than 2 when measured at ten minutes after application.

Alcohols, i.e. C1—C6 alkanols, are excluded from the inventive compositions, to avoid the drying effect on the skin. Thus, the compositions contain less than 20 wt.% of such alcohols, preferably less than 10 wt. %, more preferably less than 5 wt%, and optimally do not contain alcohols at all.

Surfactants, especially anionic and nonionic surfactants, are kept at a minimum in the inventive compositions, in order not to damage skin and also maintain the aesthetic feel of leave-on compositions. The inventive compositions preferably do not include more than 5wt.% of surfactants, preferably not more than 2wt.%. Anionic or nonionic surfactants that are limited, or preferably excluded, in the inventive compositions are as follows:

nonionic surfactants with a C10—C20 fatty alcohol or acid hydrophobe condensed with from 4 to 100 moles of ethylene oxide or propylene oxide per mole of hydrophobe; C2—C10 alkyl phenols condensed with from 4 to 20 moles of alkylene oxide; block copolymers (ethylene oxide/propylene oxide); and polyoxyethylene sorbitan as well as combinations thereof. Also, polyglycosides and saccharide fatty amides (e.g. methyl gluconamides) are preferably excluded.

anionic surfactants include soap, alkyl ether sulfate and sulfonates, alkyl sulfates and sulfonates, alkylbenzene sulfonates, alkyl and dialkyl sulfosuccinates, C8—C20 acyl isethionates, acyl glutamates, C8—C20 alkyl ether phosphates and combinations thereof.

The inventive compositions are preferably intended for use on the face and neck, since those are the areas of the skin that suffer most from the oily appearance of the skin. Thus, the inventive compositions, especially if intended for the application to the face and neck, must be aesthetically pleasing, i.e. they should not leave the white residue on the skin (as is the case with many anti-perspirant compositions. The inventive compositions are preferably essentially free (i.e. contain less than 1%, preferably less than 0.5% and most preferably less than 0.1%) of whitening anti-perspirant ingredients, such as aluminum chlorohydrate, aluminum dichlorohydrate, aluminum sesquichlorohydrate, aluminum zirconium chlorohydrate, aluminum zirconium pentachlorohydrate, aluminum zirconium octachlorohydrate, aluminum zirconium tetrachlorohydrate, aluminum zirconium trichlorohydrate.

The inventive compositions need to penetrate into the sebaceous gland, in order to act on sebocytes and provide sebum control. Thus, TCC in the inventive compositions should not be formulated so as to prevent or delay its release. For example TCC should not be encapsulated, and the composition should be essentially free of (i.e., less than 2%, preferably less than 1%, and most preferably less than 0.1%) capsule-forming materials, such as microcrystalline wax, and collagen.

The compositions according to the invention comprise a cosmetically acceptable vehicle to act as a diluant, dispersant or carrier for TCC in the composition, so as to facilitate their distribution when the composition is applied to the skin.

The vehicle may be aqueous, anhydrous or an emulsion. Preferably, the compositions are aqueous or an emulsion, especially water-in-oil or oil-in- water emulsion. Water when present will be in amounts which may range from 5 to 99%, preferably from 40 to 90%, optimally between 60 and 90% by weight.

The vehicle can also be a liposome or a niosome. A liposome or a niosome is a spherical vesicular structure consisting of one or more concentric spheres of lipid bilayers separated by water. These spherical structures can be prepared with diameters ranging from 20 nanometers to several micrometers. When phospholipids are used to make this vesicular structures, they are called liposomes and when they are prepared from nonionic surfactants they are known in the trade as niosomes.

Emollient materials may also serve as cosmetically acceptable carriers. These may be in the form of silicone oils and synthetic esters. Amounts of the emollients may range anywhere from 0.1 to 50%, preferably between 1 and 20% by weight.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from 3 to 9, preferably from 4 to 5, silicon atoms. Linear volatile silicone materials generally have viscosities less than about 5 centistokes at 25° C. while cyclic materials typically have viscosities of less than about 10 centistokes. Nonvolatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 25 million centistokes at 25° C. Among the preferred non-volatile emollients useful in the present compositions are the polydimethyl siloxanes having viscosities from about 10 to about 400 centistokes at 25° C.

Among the ester emollients are:

(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.

(2) Ether-esters such as fatty acid esters of ethoxylated fatty alcohols.

(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.

(4) Wax esters such as beeswax, spermaceti, myristyl myristate, stearyl stearate and arachidyl behenate.

(5) Sterols esters, of which cholesterol fatty acid esters are examples thereof.

Fatty acids having from 10 to 30 carbon atoms may also be included as cosmetically acceptable carriers for compositions of this invention. Illustrative of this category are pelargonic, lauric, myristic, palmitic, stearic, isostearic, hydroxystearic, oleic, linoleic, ricinoleic, arachidic, behenic and erucic acids.

Humectants of the polyhydric alcohol type may also be employed as cosmetically acceptable carriers in compositions of this invention. The humectant aids in increasing the effectiveness of the emollient, reduces scaling, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycol or sodium hyaluronate. The amount of humectant may range anywhere from 0.5 to 30%, preferably between 1 and 15% by weight of the composition.

Thickeners may also be utilized as part of the cosmetically acceptable carrier of compositions according to the present invention. Typical thickeners include crosslinked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0001 to 5%, usually from 0.001 to 1%, optimally from 0.01 to 0.5% by weight.

Collectively, the water, solvents, silicones, esters, fatty acids, humectants and/or thickeners will constitute the cosmetically acceptable carrier in amounts from 1 to 99.9%, preferably from 80 to 99% by weight.

Various types of additional active ingredients may be present in cosmetic compositions of the present invention. Actives are defined as skin benefit agents other than emollients and other than ingredients that merely improve the physical characteristics of the composition. Although not limited to this category, general examples include additional anti-sebum ingredients and sunscreens.

A preferred additional anti-sebum agent is a retinoid (i.e., retinol, retinoic acid, retinal or retinyl ester). It has been found that TCC had improved sebum suppressive activity in the presence of a retinoid.

The term “retinol” includes the following isomers of retinol: all-trans-retinol, 13-cis-retinol, 11-cis-retinol, 9-cis-retinol, 3,4-didehydro-retinol. Preferred isomers are all-trans-retinol, 13-cis-retinol, 3,4-didehydro-retinol, 9-cis-retinol. Most preferred is all-trans-retinol, due to its wide commercial availability.

Retinyl ester is an ester of retinol. The term “retinol” has been defined above.

Retinyl esters suitable for use in the present invention are C 1-C 30 esters of retinol, preferably C 2-C 20 esters, and most preferably C 2, C 3, and C 16 esters because they are more commonly available. Examples of retinyl esters include but are not limited to: retinyl palmitate, retinyl formate, retinyl acetate, retinyl propionate, retinyl butyrate, retinyl valerate, retinyl isovalerate, retinyl hexanoate, retinyl heptanoate, retinyl octanoate, retinyl nonanoate, retinyl decanoate, retinyl undecandate, retinyl laurate, retinyl tridecanoate, retinyl myristate, retinyl pentadecanoate, retinyl heptadeconoate, retinyl stearate, retinyl isostearate, retinyl nonadecanoate, retinyl arachidonate, retinyl behenate, retinyl linoleate, retinyl oleate, retinyl lactate, retinyl glycolate, retinyl hydroxy caprylate, retinyl hydroxy laurate, retinyl tartarate.

The preferred ester for use in the present invention is selected from retinyl palmitate, retinyl acetate and retinyl propionate, because these are the most commercially available and therefore the cheapest. Retinyl ester is also preferred due to its efficacy.

The retinoid is employed in the inventive composition in an amount of from about 0.001% to about 10%, preferably in an amount of from about 0.01% to about 1%, most preferably in an amount of from about 0.01% to about 0.5%.

Sunscreens include those materials commonly employed to block ultraviolet light. Illustrative compounds are the derivatives of PABA, cinnamate and salicylate. For example, avobenzophenone (Parsol 1789®) octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sunscreen employed in the compositions can vary depending upon the degree of protection desired from the sun's UV radiation.

Many cosmetic compositions, especially those containing water, must be protected against the growth of potentially harmful microorganisms. Although TCC is an anti-microbial, the inventive compositions preferably also include preservatives. Suitable preservatives include alkyl esters of p-hydroxybenzoic acid, hydantoin derivatives, propionate salts, and a variety of quaternary ammonium compounds. Particularly preferred preservatives of this invention are methyl paraben, propyl paraben, phenoxyethanol and benzyl alcohol. Preservatives will usually be employed in amounts ranging from about 0.1% to 2% by weight of the composition.

The composition according to the invention is intended primarily as a product for topical application to human skin, especially as an agent for controlling or preventing excessive sebum secretion.

In use, a quantity of the composition, for example from 1 to 100 ml, is applied to exposed areas of the skin, from a suitable container or applicator and, if necessary, it is then spread over and/or rubbed into the skin using the hand or fingers or a suitable device.

Product Form and Packaging:

The cosmetic skin composition of the invention can be in any form, e.g. formulated as a toner, gel, lotion, a fluid cream, or a cream. The composition can be packaged in a suitable container to suit its viscosity and intended use by the consumer. For example, a lotion or fluid cream can be packaged in a bottle or a roll-ball applicator or a propellant-driven aerosol device or a container fitted with a pump suitable for finger operation. When the composition is a cream, it can simply be stored in a non-deformable bottle or squeeze container, such as a tube or a lidded jar. The invention accordingly also provides a closed container containing a cosmetically acceptable composition as herein defined.

The composition may also be included in capsules such as those described in U.S. Pat. No. 5,063,057, incorporated by reference herein.

The following specific examples further illustrate the invention, but the invention is not limited thereto.

### EXAMPLE 1

This example reports an in vitro analysis of sebum suppression by various test compounds.

Secondary cultures of human sebocytes obtained from an adult male were grown in 48-well tissue culture plates (Costar Corp.; Cambridge, Mass.) until two days post-confluence. Sebocyte growth medium consisted of Clonetics Keratinocyte Basal Medium (KBM) supplemented with 14 μg/ml bovine pituitary extract, 0.4 μg/ml hydrocortisone, 5 μg/ml insulin, 10 μg/ml epidermal growth factor, 1.2×10-10 M cholera toxin, 100 units/ml penicillin, and 100 μg/ml streptomycin. All cultures were incubated at 37° C. in the presence of 7.5% CO2. Medium was changed three times per week.

On the day of experimentation, the growth medium was removed and the sebocytes washed three times with sterile Dulbecco's Modified Eagle Medium (DMEM; phenol red free). Fresh DMEM was added to each sample (triplicates) with 1-5 microliter of test agent solubilized in ethanol. Controls consisted of addition of ethanol alone. Each plate was returned to the incubator for 20 hours.

This was followed by the addition of 14C-acetate buffer (5 mM final concentration, 56 mCi/mmol specific activity). Sebocytes were returned to the incubator for four hours afterwhich each culture was rinsed three times with phosphate buffered saline to remove unbound label. Radioactive label remaining in the sebocytes was harvested and counted using a Beckman scintillation counter.

For the experiments summarized in Tables 1 and 2, lactate dehydrogenase amount was also measured. After 20 hours, 25 μl of supernatant was removed from each well and transferred to a 96-well plate. A vial of lactate dehydrogenase reagent (LD-L-20; Sigma Diagnostics; Cat 228-20) was reconstituted with 20 ml of distilled water. 200 μl of this reconstituted reagent was added to each well and the plate immediately analyzed at 340 nm. After exactly five minutes at room temperature, the plate was re-analyzed at 340 nm. The amount of lactate dehydrogenase activity was calculated by subtracting the initial reading from the five minute reading (this value indicated below in column headed “LDH”). The higher the number, the more lactate dehydrogenase is present. Increased lactate dehydrogenase numbers are associated with cellular crisis. Experimental samples were compared to the value obtained with the ethanol treated control.

The results that were obtained are summarized in Tables 1-3.

TABLE 1

| Treatment | % of Control | T-Test | LDH |
|---|---|---|---|
| Control | 100.0 | — | 0.094 |
| 1 μM TCC | 70.4 | 0.010 | 0.039 |
| 10 μM TCC | 1.0 | <0.0001 | 0.059 |
| 100 μM TCC | 0.8 | <0.0001 | 0.114 |
| 1 mM TCC | 0.8 | <0.0001 | 0.190 |

TABLE 2

| Treatment | % of Control | T-Test | LDH |
|---|---|---|---|
| Control | 100.0 | — | 0.154 |
| 100 nM TCC | 80.4 | 0.0002 | 0.137 |
| 500 nM TCC | 54.6 | 0.0003 | 0.136 |
| 1 μM TCC | 20.9 | 1.7 × 10⁻⁶ | 0.183 |
| 1 μM Phenol Red | 91.4 | 0.006 | 0.169 |
| 10 μM Phenol Red | 70.9 | 0.002 | 0.237 |

TABLE 3

| Treatment | % of Control | T-Test |
|---|---|---|
| Control | 100.0 | — |
| 250 nM TCC | 97.6 | 0.647 |
| + 1 μM Retinol | 80.7 | 0.073 |
| + 10 μM Retinol | 70.4 | 0.008 |
| 500 nM TCC | 75.6 | 0.407 |
| + 1 μM Retinol | 92.2 | 0.537 |
| + 10 μM Retinol | 53.8 | 0.110 |
| 1 μM TCC | 81.4 | 0.137 |
| + 1 μM Retinol | 34.8 | 0.193 |
| + 10 μM Retinol | 6.5 | 0.002 |
| 2.5 μM TCC | 4.3 | 0.001 |
| + 1 μM Retinol | 1.6 | 0.001 |
| + 10 μM Retinol | 1.7 | 0.001 |
| 1 μM Retinol | 103.7 | 0.716 |
| 10 μM Retinol | 101.6 | 0.386 |

It can be seen from the results in Tables 1-3 that TCC had sebum suppressive activity. As evidenced by the results in Table 2, TCC was more effective than Phenol Red (a known sebum suppressive agent). As can be seen from the results in Table 3, the sebum suppresive activity of TCC was significantly enhanced in the presence of retinol.

### EXAMPLE 2

Greasy Feel Panel Test:

In order to measure the effect of the leave-on products on the greasy feel on the face, a grease feel panel test was carried out. The test involved an expert sensory panel who are specially trained and qualified in carrying out the Grease Feel Panel Test. The methodology is fully based on Q.D.A. (Quantitative Descriptive Analysis) sensory techniques. The panel used the QDA profiling technique, which measures, analyzes and interprets the amount or intensity of a stimulus as it is perceived by the human senses.

The panel was comprised of women (18-65 years old) with a high degree of sensory acuity who were trained on sensory perceptions and scaling, so that it can be used as a fine tuned assessment “instrument”. This is a randomized, double-blind incomplete block design with restricted randomization (Latin Square) study where each subject evaluated the formulation C (hair cream) on one side and a test formulation, A, B or D (see below) on the other side. The protocol was as follows:

Approximately 0.25 ml of a formulation was dispensed to a 5.08 cm diameter weigh boat and applied on the face for 25 seconds using two fingers. The Mean Greasy Feel Value was measured by magnitude estimation for each side. Additionally, a forced choice comparison was conducted.

For the Mean Greasy Feel Value, the degree of greasiness was estimated at one and ten minutes after application using the 0-9 scale with 0 as non greasy and 9 as extreme greasy. Johnson's Baby Oil (Johnson and Johnson) was used for grade 0, mineral oil (USP/FCC; paraffin oil heavy 0122-1) from Fisher Scientific for grade 4 and Vaseline Petroleum Jelly (CheseBrough Ponds) as grade 9. In the Forced choice comparison, at one and ten minute evaluation point, the response to the forced choice assessment “Which side of the face feels more greasy” was analyzed.

An Analysis of Variance (ANOVA) model was used to evaluate the data with panelist, treatment and panelist by treatment interaction as the factors in the model. Panelist and panelist by treatment interaction was treated as random effects and, therefore, the panelists by treatment interaction was used as the error term to test the significance of treatment effects. Statistical significance was determined at p<0.1.

Base Formula A: Niosome with TCC

| Material Trade Name | Wt. % | Chemical Name | Manufacturer |
|---|---|---|---|
| Part (A): | | | |
| Santone 3-1-S XTR | 6.4 | Triglycerol | Loders |
| | | monostearate | Croklaan |
| Cholesterol CG | 3.0 | Cholesterol | Croda |
| Dihexadecyl | 0.6 | Dihexadecyl | Aldrich |
| Phosphate | | phosphate | |
| Part (B): | | | |
| Water, DI | 32 | | |
| Glucose | 4 | Glucose | Fluka |
| | | anhydrous | |
| Part (C): | | | |
| Glydant plus | 0.15 | DM Hydantoin, | Lonza |
| | | 3-iodo-2- | |
| | | propynybutyl | |
| | | Carbamate | |
| Part (D): | | | |
| Water | 48.9 | | |
| Part (E): | | | |
| Hetester PHA | 4 | Propylene glycol | Heterene, inc. |
| | | isoceteth-3 | |
| | | acetate | |
| Part (F): | | | |
| 3,4,4- | 1.0 | 3,4,4- | Aldrich |
| Trichlorocarbanilide | | Trichlorocarbanili | |
| | | de, 99% | |

Method of Preparation: Combine phase A, melt at 80° C. for 30 min. Using overhead mixer, mix at 500 rpm for 30 min. Dissolve glucose in water (B), add to phase A. Mix for 60 min at 500 rpm. Add Phase C to A+B. Add water (phase D) to A+B+C and mix at 1000 rpm for 60 min while maintaining temperature at 80C. Lower the temperature of mixture to 50C and add premixed phases E and F to it. Mix the system for 30 minutes at 500 rpm.

Base Formula B: Oil-in-water emulsion with TCC

| Material Trade Name | Wt. % | Chemical Name | Manufacturer |
|---|---|---|---|
| Part (A): | | | |
| Water, DI | To 100% | Water, DI | |
| Urea | 5.0 | Urea | Spectrum |
| 1,3-Butylene glycol | 2.0 | 1,3-Butylene | Hoechst |
| | | glycol | Celanese |
| Edicol 421 | 1.5 | Ethoxydiglycol | MMP Inc. |
| Tween 60 | 0.25 | Polysorbate 60 | ICI |
| Pationic SSL | 0.25 | Sodium stearyl | RITA Corp. |
| | | lactylate | |
| Glycerin | 0.2 | Glycerin | Dow |
| Keltrol 1000 | 0.1 | Xanthan gum | Kelco/Merck |
| Hampene Na2 | 0.075 | Disodium EDTA | Hampshire |
| Part (B): | | | |
| Span 80 | 1.58 | Sorbitan | ICI |
| | | Monooleate | |
| Dow Corning 200/10 | 1.5 | Dimethicone | Dow Corning |
| Fluid | | | |
| Span 60 | 0.66 | Sorbitan | ICI |
| | | Monostearate | |
| Gransil PM-56 | 0.5 | Phenyl | Grant |
| | | Trimethicone | |
| Xalifin 15 | 0.4 | C12-20 Acid | Vevy Europe |
| | | PEG-8 Ester | |
| Vitamin E acetate | 0.1 | Tocopheryl | BASF |
| | | acetate | |
| Part (C): | | | |
| Ethanol SDA 40B | 7.5 | Ethanol | Pharmco |
| Hetester PHA | 6 | Propylene glycol | Heterene |
| | | isoceteth-3 | |
| | | acetate | |
| Dowanol Eph | 0.4 | 2- | Dow |
| | | Phenoxyethanol | Chemical |
| 3,4,4- | 0.3 | 3,4,4- | Aldrich |
| Trichlorocarbanilide | | Trichlorocarbanili | |
| | | de, 99% | |
| Part (D): | | | |
| Micro Ace P-2 Talc | 5.0 | Talc | Presperse, Inc. |
| Microma 100 | 1.0 | Polymethylmetha | Ikeda |
| | | crylate | |
| Part (E): | | | |
| Sepigel 305 | 1.5 | Polyacrylamide | Seppic |
| | | (and) C13-14 | |
| | | Isoparaffin (and) | |
| | | Laureth-7 | |

Method of Preparation: Base formula B was prepared by first heating both Part (A) and (B) to about 70° C. and combining them, cooling down to 30 °C., then adding Part (C), (D), and (E) consecutively. Vigorous mixing is required throughout the addition of Part (C), (D), and (E).

Comparative Base Formula C: (example 16 in U.S. Pat. 4,728,667)

| Material Trade Name | Wt. % | Chemical Name | Manufacturer |
|---|---|---|---|
| Part (A): | | | |
| Carnation White Mineral | 15.0 | Mineral oil | Carnation |
| oil | | | |
| Lanette 16 | 5.0 | Cetyl alcohol | Henkel |
| Petrolium Jelly 2.5 Hard | 4.0 | Petrolatum | Witco |
| Emerest 2400 | 3.0 | Glycerol | Henkel |
| | | monostearate | |
| Brij 98 | 1.0 | Polyoxyethylene | ICI Surfactants |
| | | (20) Oleyl ether | |
| 3,4,4-Trichlorocarbanilide | 1.0 | 3,4,4- | Aldrich |
| | | Trichlorocarbanili | |
| | | de, 99% | |
| Vitamin E Acetate | 0.1 | Vitamin E | BASF |
| | | Acetate | |
| Part (B): | | | |
| Water, DI | 61.1 | Water | |
| Dipropylene glycol | 10 | Dipropylene | Sigma |
| | | glycol | |

Method of Preparation: Base formula C was prepared the following way.

First, Part (A) ingredients were heated until molten (about 75° C.). At the same time, Part (B) ingredients were heated to the same temperature as Part (A). Then, Part (A) and (B) were combined and mixed well while cooling down to room temperature.

The results that were obtained are summarized in Tables 4 and 5.

TABLE 4: Magnitude estimation of sensory attributes. Mean response at 95% confidence level; Evaluations =; Randomized Incomplete Block Design across 4 days; subjects 11-12; Attributes are rated on a 9 point scale (0=no greasiness and 9=extreme)

TABLE 4
Magnitude estimation of sensory attributes. Mean response at 95%
confidence level; Evaluations =; Randomized Incomplete Block Design
across 4 days; subjects 11-12; Attributes are rated on a 9 point scale
(0 = no greasiness and 9 = extreme)

| | | P value | | P value | |
|---|---|---|---|---|---|
| | | vs. | Base | vs. | Comparative |
| | Base | Formula | Formula | Formula | Base |
| Attribute | Formula A | C | B | C | Formula C |
| Mean Greasy | 2.54 ± 0.534 | 0.0622 | 2.61 ± | 0.0070 | 3.83 ± 0.284 |
| Feel Value one | | | 0.534 | | |
| minute after | | | | | |
| Application | | | | | |
| Mean Greasy | 1.38 ± 0.325 | 0.0209 | 1.54 ± | 0.0019 | 2.61 ± 0.173 |
| Feel Value ten | | | 0.325 | | |
| minutes after | | | | | |
| application | | | | | |

Overall, the Comparative Formulation C had the most negative greasiness attribute.

TABLE 5 : Directed difference forced choice utilizing a magnitude scale of 0-9 where 0=none and 9=extreme greasiness. Mean response at 95% confidence level. Evaluations=Randomized Incomplete Block Design across 4 day; Subjects=8. A positive score indicates Formulation C is greasier.

TABLE 5
Directed difference forced choice utilizing a magnitude scale of 0-9
where 0 = none and 9 = extreme greasiness. Mean response at 95%
confidence level. Evaluations = Randomized Incomplete Block Design
across 4 day; Subjects = 8. A positive score indicates
Formulation C is greasier.

| | Formula C | | Formula C | |
|---|---|---|---|---|
| Attribute | vs. A | P value | vs. B | P value |
| Forced Choice | 4.25 | 0.003 | 3.83 | 0.00 |
| Greasiness one minute | | | | |
| after application | | | | |
| Forced Choice | 3.50 | 0.001 | 3.17 | 0.015 |
| Greasiness ten minutes | | | | |
| after application | | | | |

It should be understood that the specific forms of the invention herein illustrated and described are intended to be representative only. Changes, including but not limited to those suggested in this specification, may be made in the illustrated embodiments without departing from the clear teachings of the disclosure. Accordingly, reference should be made to the following appended claims in determining the full scope of the invention.

What is claimed is:

1. A leave-on skin care cosmetic composition for delivery into sebaceous glands of the skin comprising:
(i) from about 0.001% to about 10% of 3,4,4-trichlocarbanilide;
(ii) not more than 20% of an alcohol;
(iii) not more than 5% of a surfactant; and
(iv) a cosmetically acceptable vehicle,
wherein the composition has a Mean Greasy Feel Value of less than about 3.5 one minute after application to the skin.

2. The composition of claim 1 wherein the composition has a Mean Greasy Feel Value of less than about 2.0 ten minutes after application to the skin.

3. The composition of claim 1, wherein the composition further comprises a retinoid.

4. A method of reducing or preventing oily skin conditions, the method comprising applying to the skin the composition of claim 1.

5. A cosmetic method of reducing or preventing sebum secretion from sebocytes, the method comprising applying to the skin the composition of claim 1.
